(19) United States
(12) Patent Application Publication
(10) Pub. No.: US 2015/0360041 A1
(43) Pub. Date: Dec. 17, 2015

(54) SYSTEMS AND METHODS FOR TREATING CARDIAC ARRHYTHMIAS

(71) Applicant: CARDIAC PACEMAKERS, INC., St. Paul, MN (US)

(72) Inventors: Jeffrey E. Stahmann, Ramsey, MN (US); Keith R. Maile, New Brighton, MN (US); Michael J. Kane, Roseville, MN (US); William J. Linder, Golden Valley, MN (US); Howard D. Simms, JR., Shoreview, MN (US)

(21) Appl. No.: 14/737,077

(22) Filed: Jun. 11, 2015

Related U.S. Application Data
(60) Provisional application No. 62/011,175, filed on Jun. 12, 2014.

(51) Int. Cl. A61N 1/39; A61N 1/362

(52) U.S. Cl. CPC A61N 1/3962; A61N 1/3621; A61N 1/3987

(57) ABSTRACT

Systems and methods for coordinating treatment of abnormal heart activity using multiple implanted devices within a patient. In one example, a leadless cardiac pacemaker (LCP) may receive signals related to one or more physiological conditions of a patient, wherein the LCP may be configured to deliver ATP therapy to a heart. The LCP may also be configured, based at least in part on the received signals, to detect an arrhythmia. In response to detecting an arrhythmia, the LCP may determine whether to deliver ATP therapy to the heart. If the LCP determines to deliver ATP therapy, the LCP may deliver ATP therapy to the heart.

### CROSS-REFERENCES TO RELATED APPLICATIONS

This application claims the benefit of U.S. Provisional Application No. 62/011,175, filed Jun. 12, 2014, the complete disclosure of which is herein incorporated by reference.

### TECHNICAL FIELD

The present disclosure generally relates to systems, devices, and methods for treating cardiac arrhythmias, and more particularly, to systems, devices, and methods for detecting cardiac arrhythmias and delivering anti-tachycardia pacing (ATP) therapy using a leadless cardiac pacemaker.

### BACKGROUND

Pacing instruments can be used to treat patients suffering from various heart conditions that may result in a reduced ability of the heart to deliver sufficient amounts of blood to a patient's body. These heart conditions may lead to rapid, irregular, and/or inefficient heart contractions. To help alleviate some of these conditions, various devices (e.g., pacemakers, defibrillators, etc.) can be implanted in a patient's body. Such devices may monitor and provide electrical stimulation to the heart to help the heart operate in a more normal, efficient and/or safe manner. In some cases, a patient may have multiple implanted devices.

### SUMMARY

The present disclosure generally relates to systems, devices, and methods for treating cardiac arrhythmias, and more particularly, to systems, devices, and methods for detecting cardiac arrhythmias and delivering anti-tachycardia pacing (ATP) therapy using a leadless cardiac pacemaker.

In one example, a leadless cardiac pacemaker (LCP) may include a housing, one or more exposed electrodes, a battery, and a processing module. The processing module may be operatively coupled to the one or more exposed electrodes and the battery. The processing module may be configured to receive signals related to one or more physiological conditions of the patient via one or more of the exposed electrodes, detect an arrhythmia based, at least in part, on the received signals, determine whether to deliver ATP therapy, deliver ATP therapy via one or more of the exposed electrodes if it is determined to deliver ATP therapy, and determine whether the delivered ATP therapy terminated the arrhythmia.

In another example, a method for delivering anti-tachycardia pacing (ATP) therapy to a heart of a patient may include: receiving signals related to one or more physiological conditions of the patient at a leadless cardiac pacemaker (LCP) that is configured to deliver ATP therapy to the heart; the LCP detecting an arrhythmia based, at least in part, on the received signals; in response to detecting the arrhythmia, the LCP determining whether to deliver ATP therapy to the heart; after determining to deliver ATP therapy, the LCP delivering ATP therapy to the heart; and after delivering ATP therapy, the LCP determining whether the delivered ATP therapy terminated the arrhythmia.

In another example, a method for delivering anti-tachycardia pacing (ATP) therapy to a heart of a patient may include: with a leadless cardiac pacemaker (LCP), determining whether to deliver ATP to the heart of the patient in response to detecting an arrhythmia, after determining to deliver ATP therapy, delivering ATP therapy with the LCP to the heart of the patient, determining whether the delivered ATP therapy terminated the arrhythmia, and if the delivered ATP therapy failed to terminate the arrhythmia, delivering one or more defibrillation shocks to the heart of the patient via an implantable medical device.

In yet another example, an implantable medical device system for delivering anti-tachycardia pacing (ATP) therapy to a heart of a patient may include a leadless cardiac pacemaker (LCP) implanted in or proximate the heart of the patient, and another implantable medical device implanted within the patient and communicatively coupled to the LCP. The LCP may be configured to sense cardiac electrical signals and determine whether to deliver ATP therapy based at least in part on the sensed cardiac electrical signals. The other implantable medical device may be configured to deliver one or more defibrillation shocks to the heart of the patient in response to receiving a trigger signal from the LCP.

In still another example, a method for delivering anti-tachycardia pacing (ATP) therapy to a heart of a patient may include determining an occurrence of an arrhythmia with a leadless cardiac pacemaker (LCP) based at least in part on cardiac events sensed by the LCP, determining whether to deliver ATP therapy to the heart of the patient by the LCP in response to the determined occurrence of an arrhythmia, delivering ATP therapy to the heart of the patient with the LCP after determining to deliver ATP therapy to the heart of the patient with the LCP, after delivering ATP therapy to the heart of the patient with the LCP, determining whether the delivered ATP therapy terminated the arrhythmia, after determining that the delivered ATP therapy failed to terminate the arrhythmia, communicating a trigger message from the LCP to an implantable cardioverter-defibrillator, and delivering one or more defibrillation pulses with the implantable cardioverter-defibrillator after receiving the trigger message from the LCP.

In a first example, a leadless cardiac pacemaker (LCP) comprises a housing, one or more exposed electrodes, a power source; and a processing module, wherein the processing module is operatively coupled to the one or more exposed electrodes and the power source, and wherein the processing module is configured to: receive signals related to one or more physiological conditions of a patient via one or more of the exposed electrodes; detect an arrhythmia of a heart of the patient based, at least in part, on the received signals; determine whether to deliver ATP therapy via one or more of the exposed electrodes in response to detecting an arrhythmia; and deliver ATP therapy to the heart via one or more of the exposed electrodes if it is determined to deliver ATP therapy.

In a second example, the LCP of the first example, wherein the processor is further configured to determine whether the delivered ATP therapy terminated the arrhythmia.

In a third example, the LCP of the first example, wherein the processing module is further configured to, after determining that the delivered ATP therapy failed to terminate the arrhythmia, communicate a trigger message to a medical device, wherein the medical device comprises one of: a leadless cardiac pacemaker; an implantable cardioverter-defibrillator; a subcutaneous implantable cardioverter-defibrillator; and an external cardioverter-defibrillator.

In a fourth example, a method for delivering anti-tachycardia pacing (ATP) therapy to a heart of a patient may comprise receiving signals related to one or more physiological conditions of the patient at a leadless cardiac pacemaker (LCP) that is configured to deliver ATP therapy to the heart; the LCP detecting an arrhythmia based, at least in part, on the received signals; in response to detecting the arrhythmia, the LCP determining whether to deliver ATP therapy to the heart; and after determining to deliver ATP therapy, the LCP delivering ATP therapy to the heart.

In a fifth example, the method of fourth example may further comprise after delivering ATP therapy, the LCP determining whether the delivered ATP therapy terminated the arrhythmia.

In a sixth example, any of the fourth or fifth examples may further comprise: if the LCP determines that the delivered ATP therapy failed to terminate the arrhythmia, the LCP communicating a trigger message from the LCP to a medical device, wherein in response to receiving the trigger message, the medical device delivering one or more defibrillation shocks to the heart.

In a seventh example, the method of any of the fourth through sixth examples, may further comprise after another medical device determines that the delivered ATP therapy failed to terminate the arrhythmia, delivering one or more defibrillation shocks to the heart with the another medical device.

In an eighth example, the method of any of the fourth through seventh examples, wherein the signals received by the LCP include an ECG signal sensed by the LCP.

In a ninth example, the method of the eighth example may further comprise processing the received ECG signal to identify one or more of an atrial rate parameter, a ventricle rate parameter, a morphology parameter, a rhythm parameter.

In a tenth example, the method of the ninth example, wherein the LCP, in determining whether to deliver ATP therapy to the heart, determines if the ventricle rate parameter is above a predetermined threshold.

In an eleventh example, the method of any of the ninth through tenth examples, wherein the LCP, in determining whether to deliver ATP therapy to the heart, determines if the ventricle rate parameter is above a predetermined threshold indicating a high heart rate, and the rhythm parameter is above a predetermined threshold indicating a sufficiently regular heart rhythm.

In a twelfth example, the method of any of the ninth through eleventh examples, wherein the LCP, in determining whether to deliver ATP therapy to the heart, determines if the morphology parameter indicates a monomorphic arrhythmia and if the ventricle rate parameter is greater than the atrial rate parameter.

In a thirteenth example, the method of any of the eighth through twelfth examples, wherein at least some of the received signals are physiological signals sensed by the LCP, and at least some of the received signals are signals communicated to the LCP from another medical device.

In a fourteenth example, the method of any of the fourth through thirteenth examples, wherein at least some of the received signals are physiological signals sensed by the LCP, and at least some of the received signals are signals communicated to the LCP from another implantable device.

In a fifteenth example, the method of the fourteenth example, wherein the another implantable device is an implantable sensor device that is in wireless communication with the LCP.

In a sixteenth example, the method of any of the fourteenth and fifteenth examples, wherein the different implantable device is a subcutaneous implantable cardioverter-defibrillator that is in wireless communication with the LCP.

In a seventeenth example, a method for delivering anti-tachycardia pacing (ATP) therapy to a heart of a patient comprises: receiving signals related to one or more physiological conditions of the patient at a leadless cardiac pacemaker (LCP) that is configured to deliver ATP therapy to the heart; the LCP detecting an arrhythmia based, at least in part, on the received signals; in response to detecting the arrhythmia, the LCP determining whether to deliver ATP therapy to the heart; and after determining to deliver ATP therapy, the LCP delivering ATP therapy to the heart.

In an eighteenth example, the method of the seventeenth example, further comprising: after delivering ATP therapy, the LCP determining whether the delivered ATP therapy terminated the arrhythmia.

In a nineteenth example, the method of any of the seventeenth and eighteenth examples may further comprise if the LCP determines that the delivered ATP therapy failed to terminate the arrhythmia, the LCP communicating a trigger message from the LCP to a medical device, wherein in response to receiving the trigger message, the medical device delivering one or more defibrillation shocks to the heart.

In a twentieth example, the method of any of the seventeenth through nineteenth examples may further comprise after another medical device determines that the delivered ATP therapy failed to terminate the arrhythmia, delivering one or more defibrillation shocks to the heart with the another medical device.

In a twenty-first example, the method of any of the seventeenth through twentieth examples, wherein the signals received by the LCP include an ECG signal sensed by the LCP.

In a twenty-second example, the method of twenty-first example, may further comprise further comprising processing the received ECG signal to identify one or more of an atrial rate parameter, a ventricle rate parameter, a morphology parameter, a rhythm parameter.

In a twenty-third example, the method of any of the twenty-first and twenty-second examples, wherein the LCP, in determining whether to deliver ATP therapy to the heart, determines if the ventricle rate parameter is above a predetermined threshold.

In a twenty-fourth example, the method of any of the seventeenth through twenty-third examples, wherein the LCP, in determining whether to deliver ATP therapy to the heart, determines if the ventricle rate parameter is above a predetermined threshold indicating a high heart rate, and the rhythm parameter is above a predetermined threshold indicating a sufficiently regular heart rhythm.

In a twenty-fifth example, the method of any of the seventeenth through twenty-fourth examples, wherein the LCP, in determining whether to deliver ATP therapy to the heart, determines if the morphology parameter indicates a monomorphic arrhythmia and if the ventricle rate parameter is greater than the atrial rate parameter

In a twenty-sixth example, the method of any of the seventeenth through twenty-fifth examples, wherein at least some of the received signals are physiological signals sensed by the LCP, and at least some of the received signals are signals communicated to the LCP from another medical device.

In a twenty-seventh example, an implantable medical device system for delivering anti-tachycardia pacing (ATP) therapy to a heart of a patient comprises a leadless cardiac pacemaker (LCP) implanted in or proximate the heart of the patient; and a medical device implanted within the patient and communicatively coupled to the LCP; wherein the LCP is configured to: sense cardiac electrical signals; determine whether to deliver ATP therapy based at least in part on the sensed cardiac electrical signals; after determining to deliver ATP therapy based at least in part on the sensed cardiac electrical signals, deliver ATP therapy to the heart of the patient; and wherein the medical device is configured to deliver one or more defibrillation shocks to the heart of the patient.

In a twenty-eighth example, the system of the twenty-seventh example, wherein the LCP, when determining whether to deliver ATP therapy, determines whether an arrhythmia having predetermined characteristics is present, and if present, determines to deliver ATP therapy.

In a twenty-ninth example, the system of the twenty-seventh example, wherein the LCP is further configured to determine whether delivered ATP therapy terminated the arrhythmia, and if not, to communicate a trigger signal to the medical device; and wherein the medical device is configured to deliver one or more defibrillation shocks to the heart of the patient in response to receiving the trigger signal from the LCP.

In a thirtieth example, the system of any of the twenty-seventh through twenty-ninth examples, wherein the medical device is further configured to: determine whether delivered ATP therapy terminated the arrhythmia; and deliver one or more defibrillation shocks to the heart of the patient in response to determining that the delivered ATP therapy failed to terminate the arrhythmia.

In a thirty-first example, the system of any of the twenty-seventh through thirtieth examples, wherein: the medical device is further configured to sense cardiac electrical signals and communicate the sensed cardiac electrical signals to the LCP; and the LCP is configured to determine to deliver ATP therapy based at least in part on cardiac electrical signals sensed by the LCP and the cardiac electrical signals communicated by the medical device.

In a thirty-second example, the system of any of the twenty-seventh through thirty-first examples, wherein the LCP is further configured to communicate sensed cardiac electrical signals to the medical device, and wherein the medical device is configured to process the received cardiac electrical signals and communicate one or more processed cardiac signals to the LCP.

In a thirty-third example, the system of any of the twenty-seventh through thirty-second examples may further comprise an external support device, wherein the LCP and implantable medical device are configured for intra-body communication, and wherein the implantable medical device and external support device are configured for extracorporeal communication, and wherein the implantable medical device relays communications from the LCP to the external support device and communications from the external support device to the LCP.

In a thirty-fourth example, the system of the thirty-third example wherein the communications comprise at least one of: LCP identification; LCP programming; real-time data; and stored data.

In a thirty-fifth example, a method for delivering anti-tachycardia pacing (ATP) therapy to a heart of a patient comprises determining an occurrence of an arrhythmia with a leadless cardiac pacemaker (LCP) based at least in part on cardiac events sensed by the LCP, determining whether to deliver ATP therapy to the heart of the patient by the LCP in response to the determined occurrence of an arrhythmia, delivering ATP therapy to the heart of the patient with the LCP after determining to deliver ATP therapy to the heart of the patient with the LCP, after delivering ATP therapy to the heart of the patient with the LCP, determining whether the delivered ATP therapy terminated the arrhythmia, after determining that the delivered ATP therapy failed to terminate the arrhythmia, communicating a trigger message from the LCP to an implantable cardioverter-defibrillator, and delivering one or more defibrillation pulses with the implantable cardioverter-defibrillator after receiving the trigger message from the LCP.

In a thirty-sixth example, a leadless cardiac pacemaker (LCP) comprises a housing; one or more exposed electrodes; a power source; and a processing module, wherein the processing module is operatively coupled to the one or more exposed electrodes and the power source, and wherein the processing module is configured to: receive signals related to one or more physiological conditions of a patient via one or more of the exposed electrodes; detect an arrhythmia of a heart of the patient based, at least in part, on the received signals; determine whether to deliver ATP therapy via one or more of the exposed electrodes in response to detecting an arrhythmia; and deliver ATP therapy to the heart via one or more of the exposed electrodes if it is determined to deliver ATP therapy.

In a thirty-seventh example, the LCP of the thirty-sixth example, wherein the processor is further configured to determine whether the delivered ATP therapy terminated the arrhythmia.

In a thirty-eighth example, the LCP of any of the thirty-sixth and thirty-seventh examples, wherein the processing module is further configured to, after determining that the delivered ATP therapy failed to terminate the arrhythmia, communicate a trigger message to a medical device, wherein the medical device comprises one of: a leadless cardiac pacemaker; an implantable cardioverter-defibrillator; a subcutaneous implantable cardioverter-defibrillator; and an external cardioverter-defibrillator.

In a thirty-ninth example, the LCP of the thirty-eighth example, wherein the trigger message directs the medical device to deliver one or more defibrillation shocks to the heart of the patient.

In a fortieth example, the LCP of any of the thirty-sixth through thirty-ninth examples, wherein the signals received by the LCP include an ECG signal sensed by the LCP.

In a forty-first example, the LCP of the fortieth example, wherein the processing module is further configured to process the received ECG signal to identify one or more of an atrial rate parameter, a ventricle rate parameter, a morphology parameter, a rhythm parameter.

In a forty-second example, the LCP of the forty-first example, wherein the processing module, to determine whether to deliver ATP therapy to the heart, is configured to determine if the ventricle rate parameter is above a predetermined threshold.

In a forty-third example, the LCP of any of the forty-first and forty-second examples, wherein the processing module, to determine whether to deliver ATP therapy to the heart, is configured to determine if the ventricle rate parameter is above a predetermined threshold indicating a high heart rate, and the rhythm parameter is above a predetermined threshold indicating a sufficiently regular heart rhythm.

In a forty-fifth example, the LCP of the forty-first example, wherein the processing module, to determine whether to deliver ATP therapy, is configured to: determine if the detected arrhythmia is monomorphic; and determine if a detected rate of ventricular contraction is greater than a detected rate of atrial contraction.

In a forty-sixth example, the LCP of any of the forty-first through forty-third examples, wherein the processing module, to determine whether to deliver ATP therapy to the heart, is configured to determine if the morphology parameter indicates a monomorphic arrhythmia and if the ventricle rate parameter is greater than the atrial rate parameter.

In a forty-seventh example, the LCP of any of the thirty-sixth through forty-sixth examples, wherein the processing module is further configured to synchronize delivery of ATP therapy to the heart with an R-wave of the ECG signal, and to deliver a series of pacing pulses at rate that is below the ventricular rate parameter.

In the forty-eighth example, the LCP of any of the thirty-sixth through forty-seventh examples, wherein at least some of the received signals are physiological signals sensed by the LCP, and at least some of the received signals are signals communicated to the LCP from a different implantable device.

In a forty-ninth example, the LCP of the forty-eighth example, wherein the different implantable device is an implantable sensor device that is in wireless communication with the LCP.

In a fiftieth example, the LCP of the forty-ninth example, wherein the implantable sensor device senses one or more of the following: acceleration, blood pressure, blood flow, and sounds.

In a fifty-first example, the LCP of the forty-eighth example, wherein the different implantable device is a subcutaneous implantable cardioverter-defibrillator that is in wireless communication with the LCP.

The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The disclosure may be more completely understood in consideration of the following description of various illustrative embodiments in connection with the accompanying drawings, in which:

FIG. 1 is a schematic block diagram of an illustrative leadless cardiac pacemaker (LCP) according to one example of the present disclosure;

FIG. 2 is a schematic block diagram of another illustrative medical device that may be used in conjunction with the LCP of FIG. 1;

FIG. 3 is a schematic diagram of an exemplary medical system that includes a leadless cardiac pacemaker (LCP) and/or other devices in communication with one another;

FIG. 4 is a schematic diagram of a system including an LCP and another medical device, in accordance with yet another example of the present disclosure;

FIG. 5 is a schematic diagram of the a system including multiple LCPs, in accordance with another example of the present disclosure;

FIGS. 6A-C are timing diagrams showing some illustrative electrical stimulation protocols in accordance with an example of the present disclosure;

FIG. 7 shows an illustrative timing diagram of electrical stimulation pulses delivered by a medical device in relation to a cardiac cycle, in accordance with an example of the present disclosure;

FIG. 8 is a flow diagram of an illustrative method that may be implemented by a medical device or medical device system, such as the illustrative medical devices and medical device systems described with respect to FIGS. 1-4; and

FIG. 9 is a flow diagram of an illustrative method that may be implemented by a medical device or medical device system, such as the illustrative medical devices and medical device systems described with respect to FIGS. 1-4.

While the disclosure is amenable to various modifications and alternative forms, specifics thereof have been shown by way of example in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

### DESCRIPTION

The following description should be read with reference to the drawings in which similar elements in different drawings are numbered the same. The description and the drawings, which are not necessarily to scale, depict illustrative embodiments and are not intended to limit the scope of the disclosure.

A normal, healthy heart induces contraction by conducting intrinsically generated electrical signals throughout the heart. These intrinsic signals cause the muscle cells or tissue of the heart to contract. This contraction forces blood out of and into the heart, providing circulation of the blood throughout the rest of the body. However, many patients suffer from cardiac conditions that affect this contractility of their hearts. For example, some hearts may develop diseased tissues that no longer generate or conduct intrinsic electrical signals. In some examples, diseased cardiac tissues conduct electrical signals at differing rates, thereby causing an unsynchronized and inefficient contraction of the heart. In other examples, a heart may generate intrinsic signals at such a low rate that the heart rate becomes dangerously low. In still other examples, a heart may generate electrical signals at an unusually high rate. In some cases such an abnormality can develop into a fibrillation state, where the contraction of the patient's heart chambers are almost completely de-synchronized and the heart pumps very little to no blood.

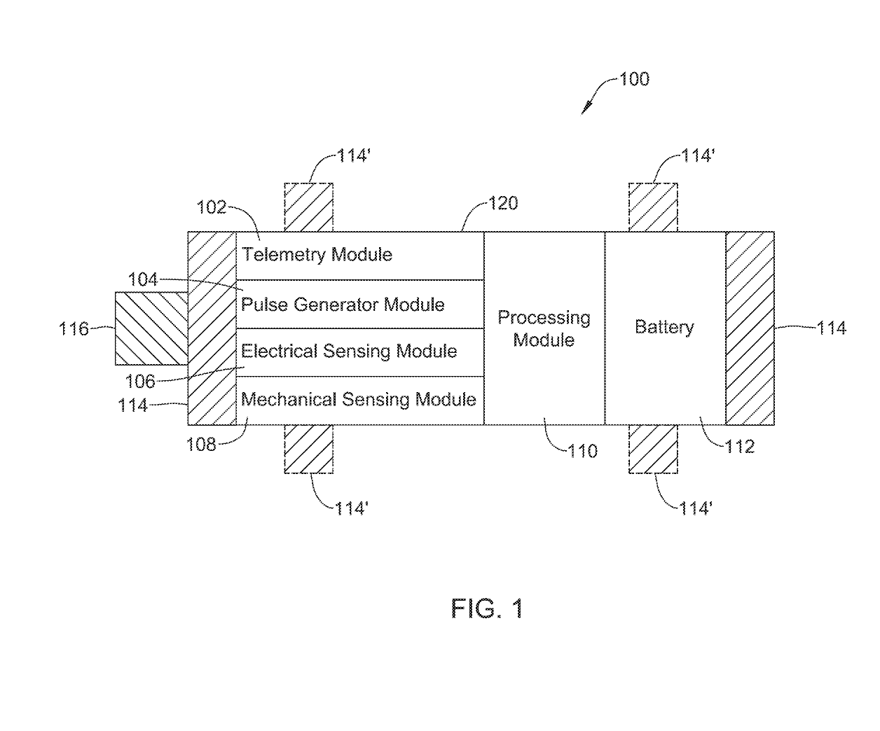

FIG. 1 depicts an exemplary leadless cardiac pacemaker (LCP) that may be implanted into a patient and may operate to prevent, control, or terminate cardiac arrhythmias in patients, for example by appropriately employing one or more therapies, or other electrical stimulation therapies known in the art (e.g. anti-tachycardia pacing (ATP) therapy, cardiac resynchronization therapy (CRT), bradycardia therapy, defibrillation pulses, or the like). As can be seen in FIG. 1, LCP 100 may be a compact device with all components housed within LCP 100 or directly on housing 120. As illustrated in FIG. 1, LCP 100 may include telemetry module 102, pulse generator module 104, electrical sensing module 106, mechanical sensing module 108, processing module 110, battery 112, and electrodes 114.

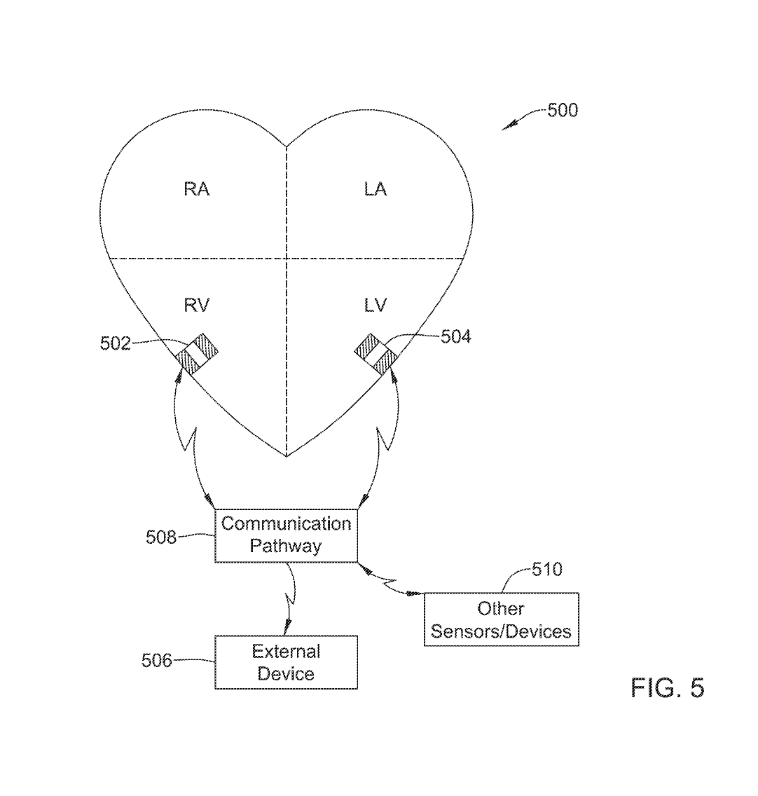

Telemetry module 102 may be configured to communicate with devices such as sensors, other medical devices, or the like, that are located externally to LCP 100. Such devices may be located either external or internal to the patient's body. Irrespective of the location, external devices (i.e. external to the LCP 100 but not necessarily external to the patient's body) can communicate with LCP 100 via telemetry module 102 to accomplish one or more desired functions. For example, LCP 100 may communicate sensed electrical signals to one or more external medical devices through telemetry module 102. The external medical devices may use the communicated electrical signals in determining occurrences of arrhythmias, delivering electrical stimulation therapy, and/or perform other functions. LCP 100 may additionally receive sensed electrical signals from one or more external medical devices through telemetry module 102, and LCP 100 may use the received sensed electrical signals in determining occurrences of arrhythmias, delivering electrical stimulation therapy, and/or performing other functions. Telemetry module 102 may be configured to use one or more methods for communicating with external devices. For example, telemetry module 102 may communicate via radiofrequency (RF) signals, inductive coupling, optical signals, acoustic signals, conducted communication signals, or any other signals suitable for communication. Some illustrative communication techniques between LCP 100 and external devices will be discussed in further detail with reference to FIG. 5 below.

In the example shown in FIG. 1, pulse generator module 104 may be electrically connected to electrodes 114. In some examples, LCP 100 may additionally include electrodes 114′. In such examples, pulse generator 104 may additionally be electrically connected to electrodes 114′. Pulse generator module 104 may be configured to generate electrical stimulation signals. For example, pulse generator module 104 may generate electrical stimulation signals by using energy stored in battery 112 within LCP 100 and deliver the generated electrical stimulation signals via electrodes 114 and/or 114′. In at least some examples, pulse generator 104 of LCP 100 may further include switching circuitry to selectively connect one or more of electrodes 114 and/or 114′ to pulse generator 104 in order to select via which electrodes 114/114′ (and/or other electrodes) pulse generator 104 delivers the electrical stimulation therapy. Pulse generator module 104 may generate electrical stimulation signals with particular features or in particular sequences in order to provide one or multiple of a number of different stimulation therapies. For example, pulse generator module 104 may be configured to generate electrical stimulation signals to provide electrical stimulation therapy to combat bradycardia, tachycardia, cardiac synchronization, bradycardia arrhythmias, tachycardia arrhythmias, fibrillation arrhythmias, cardiac synchronization arrhythmias and/or any other suitable electrical stimulation therapy.

In some examples, LCP 100 may include electrical sensing module 106, and in some cases, mechanical sensing module 108. Electrical sensing module 106 may be configured to sense the cardiac electrical activity of the heart. For example, electrical sensing module 106 may be connected to electrodes 114/114′, and electrical sensing module 106 may be configured to receive cardiac electrical signals conducted through electrodes 114/114′. For instance, the cardiac electrical signals may represent local information from the chamber in which LCP 100 is implanted. For instance, if LCP 100 is implanted within a ventricle of the heart, cardiac electrical signals sensed by LCP 100 through electrodes 114/114′ may represent ventricular cardiac electrical signals. Mechanical sensing module 108 may include, or be electrically connected to, one or more sensors, such as an accelerometer, a blood pressure sensor, a heart sound sensor, a blood-oxygen sensor, and/or other sensors which are configured to measure one or more physiological parameters of the patient. Both electrical sensing module 106 and mechanical sensing module 108 may further be connected to processing module 110 and provide signals representative of the sensed electrical activity or physiological parameters to processing module 110. Although described with respect to FIG. 1 as separate sensing modules, in some cases, electrical sensing module 206 and mechanical sensing module 208 may be combined into a single sensing module.

Processing module 110 can be configured to control the operation of LCP 100. For example, processing module 110 may be configured to receive electrical signals from electrical sensing module 110. Based on the received signals, processing module 110 may determine, for example, occurrences and, in some cases, types of arrhythmias. Based on any determined arrhythmias, processing module 110 may control pulse generator module 104 to generate electrical stimulation in accordance with one or more therapies to treat the determined arrhythmia(s). Processing module 110 may further receive information from telemetry module 102. In some examples, processing module 110 may use such received information to help determine whether an arrhythmia is occurring, determine a type of arrhythmia, and/or to take particular action in response to the information. Processing module 110 may additionally control telemetry module 108 to send information to other devices.

In some examples, processing module 110 may include a pre-programmed chip, such as a very-large-scale integration (VLSI) chip or an application specific integrated circuit (ASIC). In such embodiments, the chip may be pre-programmed with control logic in order to control the operation of LCP 100. By using a pre-programmed chip, processing module 110 may use less power than other programmable circuits while able to maintain basic functionality, thereby potentially increasing the battery life of LCP 100. In other examples, processing module 110 may include a programmable microprocessor. Such a programmable microprocessor may allow a user to adjust the control logic of LCP 100 even after implantation, thereby allowing for greater flexibility of LCP 100 than when using a pre-programmed ASIC. In some examples, processing module 110 may further include a memory circuit, and processing module 110 may store information on and read information from the memory circuit. In other examples, LCP 100 may include a separate memory circuit (not shown) that is in communication with processing module 110, such that processing module 110 may read and write information to and from the separate memory circuit.

Battery 112 may provide a power to the LCP 100 for its operations. In some examples, battery 112 may be a non-rechargeable lithium-based battery. In other examples, a non-rechargeable battery may be made from other suitable materials known in the art. Because LCP 100 is an implantable device, access to LCP 100 may be limited after implantation. Accordingly, it is desirable to have sufficient battery capacity to deliver therapy over a period of treatment such as days, weeks, months, years or decades. In other examples, battery 110 may a rechargeable lithium-based battery, which may help increase the useable lifespan of LCP 100.

As depicted in FIG. 1, LCP 100 may include electrodes 114, which can be secured relative to housing 120 but exposed to the tissue and/or blood surrounding LCP 100. In some cases, electrodes 114 may be generally disposed on either end of LCP 100 and may be in electrical communication with one or more of modules 102, 104, 106, 108, and 110. Electrodes 114 may be supported by the housing 120. In some examples, electrodes 114 may be connected to housing 120 only through short connecting wires such that electrodes 114 are not directly secured relative to housing 120.

In some examples, LCP 100 may additionally include one or more electrodes 114′. Electrodes 114′ may be positioned on the sides of LCP 100 and may increase the number of electrodes by which LCP 100 may sense cardiac electrical activity, deliver electrical stimulation and/or communicate with an external medical device. Electrodes 114 and/or 114′ can be made up of one or more biocompatible conductive materials such as various metals or alloys that are known to be safe for implantation within a human body. In some instances, electrodes 114 and/or 114′ connected to LCP 100 may have an insulative portion that electrically isolates the electrodes 114 from adjacent electrodes, housing 120, and/or other materials.

To implant LCP 100 inside a patient's body, an operator (e.g., a physician, clinician, etc.), may fix LCP 100 to the cardiac tissue of the patient's heart. To facilitate fixation, LCP 100 may include one or more anchors 116. Anchor 116 may include any one of a number of fixation or anchoring mechanisms. For example, anchor 116 may include one or more pins, staples, threads, screws, helix, tines, and/or the like. In some examples, although not shown, anchor 116 may include threads on its external surface that may run along at least a partial length of anchor 116. The threads may provide friction between the cardiac tissue and the anchor to help fix the anchor 116 within the cardiac tissue. In other examples, anchor 116 may include other structures such as barbs, spikes, or the like to facilitate engagement with the surrounding cardiac tissue.

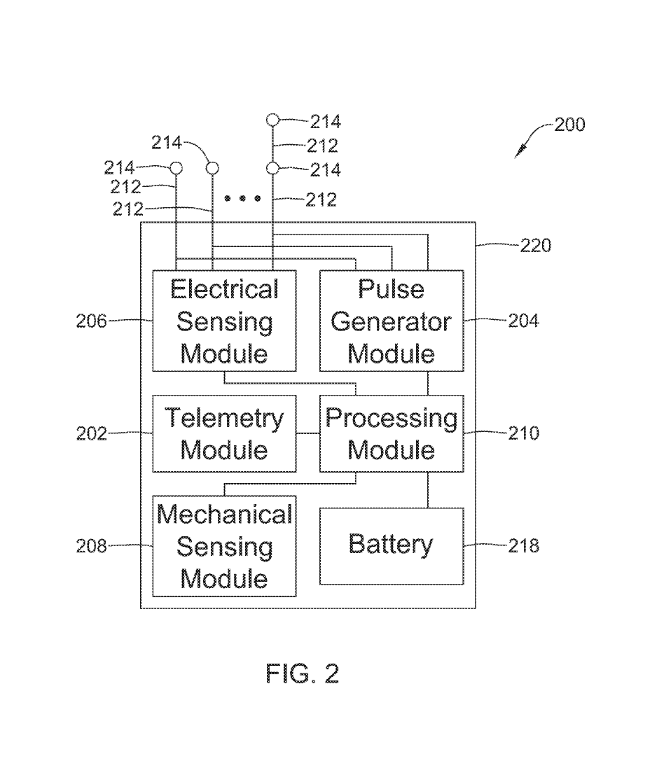

FIG. 2 depicts an example of another device, medical device (MD) 200, which may be used in conjunction with LCP 100 of FIG. 1 in order to detect and treat cardiac arrhythmias and other heart conditions. In the example shown, MD 200 may include a telemetry module 202, a pulse generator module 204, an electrical sensing module 206, a mechanical sensing module 208, a processing module 210, and a battery 218. Each of these modules may be similar to modules 102, 104, 106, 108, and 110 of LCP 100. Additionally, battery 218 may be similar to battery 112 of LCP 100. In some examples, MD 200 may have a larger volume within housing 220. In such examples, MD 200 may include a larger battery and/or a larger processing module 210 capable of handling more complex operations than processing module 110 of LCP 100.

While MD 200 may be another leadless device such as shown in FIG. 1, in some instances MD 200 may includes leads such as leads 212. Leads 212 may include electrical wires that conduct electrical signals between electrodes 214 and one or more modules located within housing 220. In some cases, leads 212 may be connected to and extend away from housing 220 of MD 200. In some examples, leads 212 are implanted on, within or adjacent to a heart of a patient. Leads 212 may contain one or more electrodes 214 positioned at various locations on leads 212 and various distances from housing 220. Some leads 212 may only include a single electrode 214, while other leads 212 may include multiple electrodes 214. Generally, electrodes 214 are positioned on leads 212 such that when leads 212 are implanted within the patient, one or more of the electrodes 214 are positioned to perform a desired function. In some cases, the one or more of the electrodes 214 may be in contact with the patient's cardiac tissue. In some cases, electrodes 214 may conduct intrinsically generated electrical signals to leads 212, e.g. signals representative of intrinsic cardiac electrical activity. Leads 212 may, in turn, conduct the received electrical signals to one or more of the modules 202, 204, 206, and 208 of MD 200. In some cases, MD 200 may generate electrical stimulation signals, and leads 212 may conduct the generated electrical stimulation signals to electrodes 214. Electrodes 214 may then conduct the electrical signals to the cardiac tissue of the patient.

While not required, in some examples MD 200 may be an implantable medical device. In such examples, housing 220 of MD 200 may be implanted in, for example, a transthoracic region of the patient. Housing 220 may generally include any of a number of known materials that are safe for implantation in a human body and may, when implanted, hermetically seal the various components of MD 200 from fluids and tissues of the patient's body.

In some cases, MD 200 may be an implantable cardiac pacemaker (ICP). In this example, MD 200 may have one or more leads, for example leads 212, which are implanted on or within the patient's heart. The one or more leads 212 may include one or more electrodes 214 that are in contact with cardiac tissue and/or blood of the patient's heart. MD 200 may be configured to sense intrinsically generated cardiac electrical signals and determine, for example, one or more cardiac arrhythmias based on analysis of the sensed signals. MD 200 may be configured to deliver CRT, ATP therapy, bradycardia therapy, and/or other therapy types via leads 212 implanted within the heart. In some examples, MD 200 may additionally be configured provide defibrillation therapy.

In some instances, MD 200 may be an implantable cardioverter-defibrillator (ICD). In such examples, MD 200 may include one or more leads implanted within a patient's heart. MD 200 may also be configured to sense cardiac electrical signals, determine occurrences of tachyarrhythmias based on the sensed signals, and may be configured to deliver defibrillation therapy in response to determining an occurrence of a tachyarrhythmia. In other examples, MD 200 may be a subcutaneous implantable cardioverter-defibrillator (S-ICD). In examples where MD 200 is an S-ICD, one of leads 212 may be a subcutaneously implanted lead. In at least some examples where MD 200 is an S-ICD, MD 200 may include only a single lead which is implanted subcutaneously, but this is not required.

In some examples, MD 200 may not be an implantable medical device. Rather, MD 200 may be a device external to the patient's body, and may include skin-electrodes that are placed on a patient's body. In such examples, MD 200 may be able to sense surface electrical signals (e.g. cardiac electrical signals that are generated by the heart or electrical signals generated by a device implanted within a patient's body and conducted through the body to the skin). In such examples, MD 200 may be configured to deliver various types of electrical stimulation therapy, including for example defibrillation therapy.

In some cases, leads 212 may contain one or more sensors, such as accelerometers, blood pressure sensors, heart sound sensors, blood-oxygen sensors, and/or other sensors which are configured to measure one or more physiological parameters of the heart and/or patient. In such examples, electrical and/or mechanical sensing module(s) 206, 208 may be in electrical communication with leads 212 and may receive signals generated from such sensors.

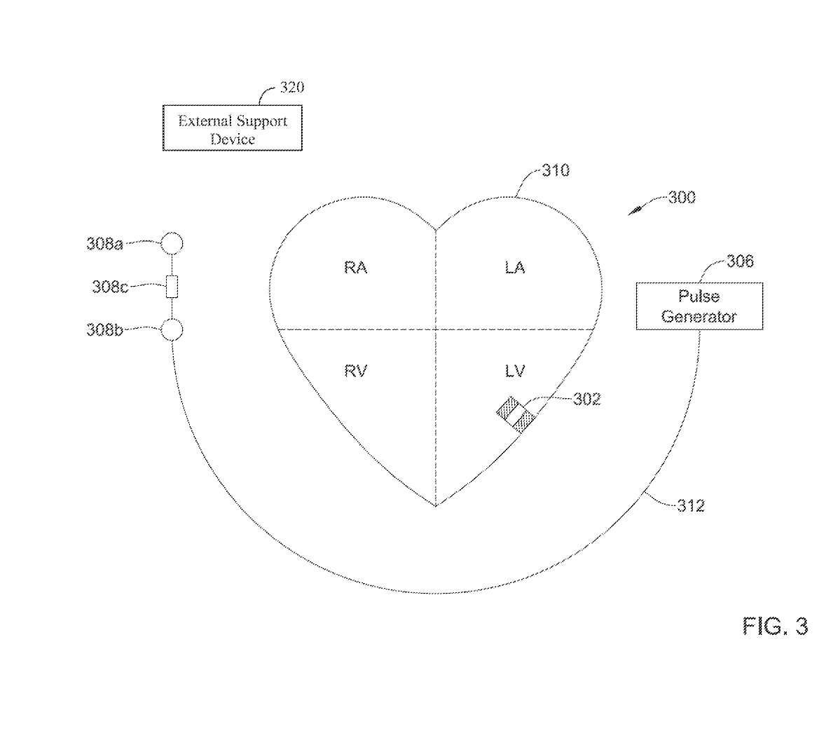

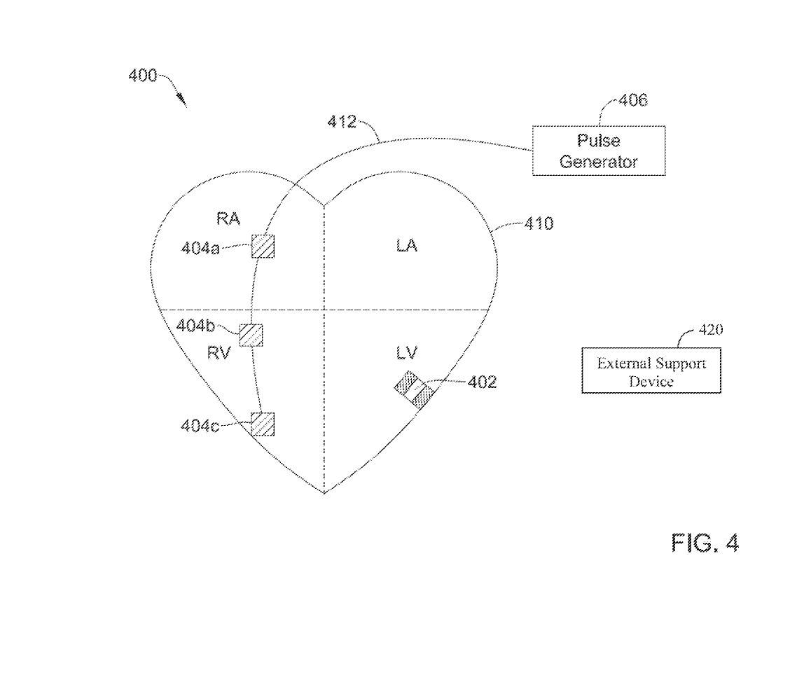

FIGS. 3 and 4 show illustrative medical device systems that may be configured to operate according to techniques disclosed herein. In FIG. 3, an LCP 302 is shown fixed to the interior of the left ventricle of the heart 310, and a pulse generator 306 is shown coupled to a lead 312 having one or more electrodes 308a-308c. In some cases, the pulse generator 306 may be part of a subcutaneous implantable cardioverter-defibrillator (S-ICD), and the one or more electrodes 308a-308c may be positioned subcutaneously adjacent the heart. In some cases, the LCP 302 may communicate with the subcutaneous implantable cardioverter-defibrillator (S-ICD).

In FIG. 4, an LCP 402 is shown fixed to the interior of the left ventricle of the heart 410, and a pulse generator 406 is shown coupled to a lead 412 having one or more electrodes 404a-404c. In some cases, the pulse generator 406 may be part of an implantable cardiac pacemaker (ICP) and/or an implantable cardioverter-defibrillator (ICD), and the one or more electrodes 404a-404c may be positioned in the heart 410. In some cases, the LCP 402 may communicate with the implantable cardiac pacemaker (ICP) and/or an implantable cardioverter-defibrillator (ICD).

The medical device systems 300 and 400 may also include an external support device, such as external support devices 320 and 420. External support devices 320 and 420 can be used to perform functions such as device identification, device programming and/or transfer of real-time and/or stored data between devices using one or more of the communication techniques described herein. As one example, communication between external support device 320 and the pulse generator 306 is performed via a wireless mode, and communication between the pulse generator 306 and LCP 302 is performed via a conducted mode. In some examples, communication between the LCP 302 and external support device 320 is accomplished by sending communication information through the pulse generator 306. However, in other examples, communication between the LCP 302 and external support device 320 may be direct.

Additionally, FIGS. 3-4 are only two examples of medical device systems that may be configured to operate according to techniques disclosed herein. Other example medical device systems may include additional or different medical devices and/or configurations. For instance, other medical device systems that are suitable to operate according to techniques disclosed herein may include additional LCPs implanted within the heart. In yet other examples, the configuration or placement of the medical devices, leads, and/or electrodes may be different from those depicted in FIGS. 3 and 4. It should be recognized that numerous other medical device systems, different from those depicted in FIGS. 3 and 4, may be operated in accordance with techniques disclosed herein. As such, the examples shown in FIGS. 3 and 4 should not be viewed as limiting in any way.

FIG. 5 illustrates an example of a medical device system and a communication pathway via which multiple medical devices may communicate. In the example shown, medical device system 500 may include LCPs 502 and 504, external medical device 506, and other sensors/devices 510. External device 506 may be any of the devices described previously with respect to MD 200. Other sensors/devices 510 may also be any of the devices described previously with respect to MD 200. In other examples, other sensors/devices 510 may include a sensor, such as an accelerometer or blood pressure sensor, or the like. In still other examples, other sensors/devices 510 may include an external programmer device that may be used to program one or more devices of system 500.

Various devices of system 500 may communicate via communication pathway 508. For example, LCPs 502 and/or 504 may sense intrinsic cardiac electrical signals and may communicate such signals to one or more other devices 502/504, 506, and 510 of system 500 via communication pathway 508. In one example, one or more of devices 502/504 may receive such signals and, based on the received signals, determine an occurrence of an arrhythmia. In some cases, device or devices 502/504 may communicate such determinations to one or more other devices 506 and 510 of system 500. Additionally, one or more of devices 502/504, 506, and 510 of system 500 may take action based on the communicated determination of an arrhythmia, such as by delivering a suitable electrical stimulation. It is contemplated that communication pathway 508 may communicate using RF signals, inductive coupling, optical signals, acoustic signals, or any other signals suitable for communication.

In some cases, communication pathway 508 communicates using conducted communication. Accordingly, devices of system 500 may have components that allow for such conducted communication. For instance, the devices of system 500 may be configured to transmit conducted communication signals (e.g. pulses) into the patient's body via one or more electrodes of a transmitting device, and may receive the conducted communication signals (e.g. pulses) via one or more electrodes of a receiving device. The patient's body may conduct the conducted communication signals (e.g. pulses) from the one or more electrodes of the transmitting device to the one or more electrodes of the receiving device in the system 500. In such examples, the delivered conducted communication signals (e.g. pulses) may differ from pacing or other therapy signals. For example, the devices of system 500 may deliver electrical communication pulses at an amplitude/pulse width that is sub-threshold to the heart. In some cases, the amplitude/pulse width of the delivered electrical communication pulses may be above the capture threshold of the heart, but may be delivered during a refractory period of the heart and/or may be incorporated in or modulated onto a pacing pulse, if desired.

In some cases, delivered electrical communication pulses may be modulated in any suitable manner to encode communicated information. In some cases, the communication pulses may be pulse width modulated. Alternatively, or in addition, the time between pulses may be modulated to encode desired communicated information. In some cases, conducted communication pulses may be voltage pulses, current pulses, biphasic voltage pulses, biphasic current pulses, or any other suitable electrical pulse as desired.

As described above, one or more medical devices may cooperate to detect cardiac arrhythmias and deliver therapy. Although the descriptions below include a medical device system including LCP 100 and MD 200, it should be understood that different medical device systems having different constituent parts may be implemented. For example, other medical device systems may include additional devices, such as additional LCPs or MDs. Still other systems may include a first LCP, such as that described above with respect to LCP 100, and a second LCP that additionally includes an ability to deliver defibrillation therapy and/or ATP therapy. Accordingly, the specific devices used in the description below are merely used as examples of medical device systems that are suitable for implementing the described techniques and should not be viewed as limiting the implementation of the described techniques to only the specifically disclosed medical device systems.

In one example, a medical device system may include an LCP device and another MD implanted within a patient, such as LCP 100 and MD 200 described above. In some examples, LCP 100 may autonomously determine when a cardiac arrhythmia is occurring. For instance, LCP 100 may determine an occurrence of a cardiac arrhythmia, and in some cases, may deliver therapy without receiving information or instructions from another device, such as MD 200. In such an example, LCP 100 may sense cardiac electrical signals via its own electrodes 114.

Using the sensed cardiac electrical signals, LCP 100 may analyze the signals to determine whether the patient is suffering from one or more cardiac arrhythmias, such as tachyarrhythmia. LCP 100 may process the received cardiac electrical signals to determine a heart rate. LCP 100 may employ any number of filtering processes to help process the received cardiac electrical signals. LCP 100 may additionally employ, for example, a peak detector or a QRS detector in order to help determine a beat rate of the patient's heart. In some cases, LCP 100 may compare the determined beat rate to one or more thresholds. Based on whether the beat rate falls above or below one or more of the thresholds, LCP 100 may determine that the patient is suffering from a cardiac arrhythmia. For example, if LCP 100 determines that the beat rate is above a tachyarrhythmia threshold, LCP 100 may determine that the patient is suffering from a tachyarrhythmia. In some examples, the tachyarrhythmia threshold may be 150 beats per minute (bpm), 165 bpm, 180 bpm, or any other suitable beat rate. In another example, LCP may determine that the beat rate is below a bradycardia threshold and, accordingly, may determine that the patient is suffering from bradycardia. In some examples, the bradycardia threshold is 60 bpm, 55 bpm, 50 bpm, or any other suitable beat rate.

In some examples, LCP 100 may additionally receive other signals representative of the physiological condition of the patient, such as signals from sensors connected, either electrically or communicatively, to LCP 100. For instance, LCP 100 may receive signals related to blood pressure, blood flow, acoustics, and/or motion, to name a few. In such examples, LCP 100 may use these additional received signals to help in determining occurrences of cardiac arrhythmias. As one example, LCP 100 may determine additional beat rate(s) based on one or more of the received blood pressure, blood flow, acoustics, and motion signals. LCP 100 may compare the one or more determined additional beat rate(s) with the beat rate determined based on the cardiac electrical signals. In situations where the one or more determined additional beat rate(s) differ by a predetermined margin from the beat rate determined based on the cardiac electrical signals, LCP 100 may determine an average beat rate, and use the average beat rate in comparisons to the thresholds. In other examples, LCP 100 may use the slowest determined beat rate for comparison to the thresholds, or use a voting scheme to identify an appropriate beat rate. Such a voting scheme may encompass determining a beat rate based on each of the received signals and choosing the mean or median beat rate. However, other voting schemes may be used.

In examples where LCP 100 has determined that a beat rate is above the tachyarrhythmia threshold, indicating that the patient is suffering from a tachyarrhythmia, LCP 100 may determine to deliver ATP therapy to the patient. In one example, if LCP 100 determines that the beat rate is above a tachyarrhythmia threshold, but below a fibrillation threshold, LCP 100 may determine to deliver ATP therapy to the patient's heart. If LCP 100 determines that the beat rate is above both the tachyarrhythmia threshold and the fibrillation threshold, LCP 100 may communicate a message immediately to MD 200 to deliver a defibrillation pulse.

In other examples, after determining that the beat rate is above the tachyarrhythmia threshold, but below a fibrillation threshold, LCP 100 may also determine whether the tachyarrhythmia is likely to be susceptible to ATP therapy before determining to deliver ATP therapy. For example, LCP 100 may determine a regularity parameter based on the cardiac electrical signals. The regularity parameter may be a measure of the regularity of the heart beats over time, and may be generated by monitoring the time between successive detected beats over a predetermined time period. If the regularity parameter is below a regularity threshold (sufficiently regular), LCP 100 may determine that the tachyarrhythmia is likely to be susceptible to ATP therapy, and may initiate ATP therapy. If the regularity parameter is above the regularity threshold (not sufficiently regular), LCP 100 may determine that the tachyarrhythmia is not likely to be susceptible to ATP therapy, and LCP 100 may communicate a message immediately to MD 200 to deliver a defibrillation pulse.

In some cases, LCP 100 may determine a morphology parameter for a tachyarrhythmia, for example, after determining a beat rate above a tachyarrhythmia threshold. The morphology parameter may indicate a difference in signal morphology of detected QRS complexes relative to one or more morphology templates. If LCP 100 determines that the difference in signal morphology of detected QRS complexes relative to the one or more morphology templates is greater than a morphology threshold, LCP 100 may determine that the tachyarrhythmia is likely to be susceptible to ATP therapy, and may deliver ATP therapy. Although, in other examples, LCP 100 may determine to deliver ATP therapy if the morphology parameter is below the morphology threshold, depending on the one or more morphology templates. In any case, if LCP 100 determines not to deliver ATP therapy, LCP may send a communication to MD 200 instructing MD 200 to deliver a defibrillation pulse.

In some instances, LCP 100 may have access to both atrial cardiac electrical signals and ventricular cardiac electrical signals. For instance, LCP 100 may have multi-sensing capabilities, where LCP 100 is able to identify signals representative of ventricular contraction and signals representative of atrial contraction. In other examples, LCP 100 may receive information relating to contraction of other heart chambers (e.g. heart chambers other than the heart chamber within which LCP 100 is implanted) from one or more other devices, such as other LCPs or other medical devices. When so provided, LCP 100 may determine whether the ventricular beat rate is greater than the atrial beat rate. If LCP 100 determines that the ventricular beat rate is greater than the atrial beat rate, perhaps by a threshold amount, the LCP 100 may determine that the tachyarrhythmia is likely to be susceptible to ATP therapy, and may initiate ATP therapy. If LCP 100 determines that the ventricular beat rate is not greater than the atrial beat rate, perhaps by a threshold amount, the LCP 100 may determine that the tachyarrhythmia is not likely to be susceptible to ATP therapy, and may communicate a message immediately to MD 200 to deliver a defibrillation pulse.

In cases where the LCP 100 determines that the detected tachyarrhythmia is likely to be susceptible to ATP therapy, the LCP 100 may be configured to deliver ATP therapy. In some cases, the LCP 100 may only determine to deliver ATP therapy after determining that any two or all of the regularity parameter, the morphology parameter, and the ventricular beat rate compared to the atrial beat rate, indicate that the tachyarrhythmia is likely to be susceptible to ATP therapy. In still other examples, LCP 100 may use other parameters in helping to determining whether the tachyarrhythmia is likely to be susceptible to ATP therapy.

After determining to deliver ATP therapy, LCP 100 may deliver ATP therapy to the patient's heart. After delivering the ATP therapy, LCP 100 may determine whether the therapy terminated the tachyarrhythmia. If the ATP therapy terminated the ATP therapy, LCP 100 may return to a monitoring status, looking for further arrhythmias. If LCP 100 determines that the ATP therapy did not terminate the tachyarrhythmia, LCP 100 may send a communication (e.g. trigger) to MD 200 instructing MD 200 to deliver a defibrillation pulse in order to attempt to terminate the tachyarrhythmia. Alternatively, LCP 100 may send a communication to another LCP such that the other LCP might also attempt ATP as delivering ATP at a different cardiac site might terminate the tachyarrhythmia. In situations where LCP 100 determines that the tachyarrhythmia is likely not susceptible to ATP therapy, LCP 100 may not deliver ATP therapy but instead send a communication to MD 200 instructions MD 200 to deliver a defibrillation pulse.

In some cases, LCP 100 may use prior ATP success or failure as criteria for determining whether to deliver ATP therapy. For example, LCP 100 may identify a tachyarrhythmia according to a number of parameters, such as beat rate, signal morphology, differing rates between atrial contraction and ventricular contraction, as explained in more detail below. If tachyarrhythmias identified with similar parameters were successfully terminated via ATP during past tachyarrhythmias, then LCP 100 may determine to deliver ATP. However, if tachyarrhythmias identified with similar parameters were not successfully terminated via ATP during past tachyarrhythmias, then LCP 100 may determine not to deliver ATP. Instead LCP 100 may send a communication (e.g. trigger) to MD 200 instructing MD 200 to deliver a defibrillation pulse in order to attempt to terminate the tachyarrhythmia.

In some examples, LCP 100 may send a communication to MD 200 to begin charging one or more capacitors of the MD 200 in preparation for delivering a defibrillation pulse before communicating an instruction to deliver the defibrillation pulse. For instance, in some systems, it may take MD 200 a number of seconds to charge one or more capacitors for delivering a defibrillation pulse from the time MD 200 receives instructions to deliver a defibrillation pulse. Accordingly, LCP 100 may send a communication to MD 200 instructing MD 200 to begin charging one or more capacitors after determining an occurrence of tachyarrhythmia and: (1) while delivering ATP therapy; and/or (2) before determining whether the tachyarrhythmia is likely to be susceptible to ATP therapy. After delivering ATP therapy and determining that the ATP therapy was unsuccessful, the LCP 100 may be configured to communicate a message to MD 200 to deliver a defibrillation pulse. In such examples, MD 200 may deliver a defibrillation pulse more quickly after receiving a message to deliver a defibrillation pulse than in examples where LCP 100 does not previously communicate a message to begin charging the one or more capacitors of the MD 200.

In still other examples, LCP 100 may send a message to MD 200 to deliver a defibrillation pulse after determining an occurrence of a tachyarrhythmia but before LCP 100 determines whether the tachyarrhythmia is likely to be susceptible to ATP therapy. The LCP 100 may then determine whether the tachyarrhythmia is likely to be susceptible to ATP therapy, and if so, delivering ATP therapy. If the delivered ATP therapy is determined to be successful in terminating the tachyarrhythmia, the LCP 100 may send a second message to MD 200 instructing the MD 200 to terminate the delivery of the defibrillation pulse. Since the time to charge the one or more capacitors of the MD 200 may take longer than determining whether the tachyarrhythmia is likely to be susceptible to ATP therapy, and if so, delivering the ATP therapy, the second message may terminate the defibrillation pulse before it is actually delivered by MD 200. However, in situations when delivery of a defibrillation pulse is actually required, the time between when the LCP 100 determines that the tachyarrhythmia is not likely to be susceptible to ATP therapy or when delivery of ATP therapy is determined to not be successful in terminating the tachyarrhythmia and when the defibrillation pulse is actually delivered may be reduced.

When LCP 100 does deliver ATP therapy, one ATP therapy protocol may include delivering electrical stimulation pulses to the heart at a rate that is faster than the intrinsically generated heart rate signals. Although this may temporarily cause the heart to beat faster, such a stimulation protocol may cause the heart to contract in response to the delivered pacing pulses as opposed to the intrinsically generated signals. The ATP therapy protocol may then slow down the rate of the delivered pacing pulses, or discontinue delivery of pacing pulses, thereby reducing the heart rate to a lower, safer level.

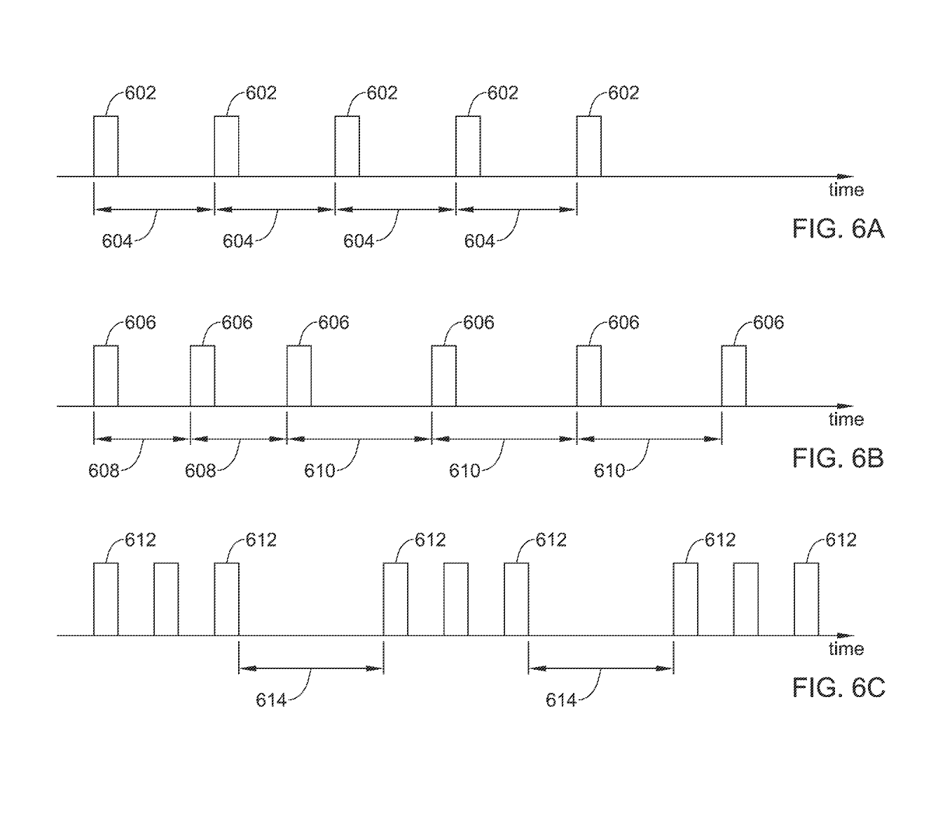

In some examples, the ATP therapy protocol may include delivery of electrical stimulation pulses at a consistent rate over a predetermined period of time or for a predetermined number of pulses. FIG. 6A depicts such a protocol with electrical stimulation pulses 602 delivered at evenly spaced intervals 604. In some instances, the rate may be determined as a fraction of the beat rate (and in some examples, specifically, the ventricular beat rate). In other examples, the ATP therapy protocol may include delivery of electrical stimulation pulses at differing rates over a predetermined period of time. For example, LCP 100 may deliver a number of electrical stimulation pulses at a first rate for a first period of time, and at a second rate for a second period of time. In another example LCP 100 may deliver electrical stimulation pulses at an ever decreasing interval for a predetermined period of time. FIG. 6B depicts a protocol with electrical stimulation pulses 606 being delivered at a first interval rate 608 followed by a second interval rate 610. In other examples, the ATP therapy protocol may include delivery of one or more series of electrical stimulation pulses with breaks between the series. FIG. 6C depicts an example of such a protocol with electrical stimulation pulses 612 being delivered in a series of three pulses, with breaks 614 between each series of three pulses. In some cases, during breaks 614, LCP 100 may determine whether a detected tachyarrhythmia has been terminated. If the tachyarrhythmia has not been terminated, LCP 100 may continue to deliver electrical stimulation pulses according to the protocol for a period of time. If the tachyarrhythmia has terminated, LCP 100 may cease delivery of the ATP therapy. It is contemplated that ATP therapy protocols may include features from any combination of those depicted in FIGS. 6A-6C. However, it should be recognized that the ATP therapy protocols shown in FIGS. 6A-6C are only illustrative, and it is contemplated that any suitable ATP therapy protocol may be used.

In some cases, LCP 100 may use a particular ATP therapy protocol based on the regularity parameter, the morphology parameter, whether the ventricular beat rate is greater than the atrial beat rate, and/or any other suitable parameter(s). For instance, if LCP 100 determines that the regularity parameter indicates that the tachyarrhythmia is likely to be susceptible to ATP therapy, but the other indicators do not indicate that the tachyarrhythmia is likely to be susceptible to ATP therapy, LCP 100 may deliver ATP therapy according to a first ATP therapy protocol. In other situations where LCP 100 determines that the morphology parameter indicates that the tachyarrhythmia is likely to be susceptible to ATP therapy, but the other indicators do not, LCP 100 may deliver ATP therapy according to a second ATP therapy protocol. These are just two examples. In other examples, LCP 100 may include logic to determine which of a number of possible ATP therapy protocols to use in response to various parameters.

LCP 100 may be preprogrammed with specific logic and therapy protocols. However, in other examples, LCP 100 may receive specific ATP therapy protocols, sometimes downloaded from MD 200 using the communication pathway 508. Aside from the rate or specific sequence of delivering electrical stimulation pulses, an ATP therapy protocol may specify parameters for the electrical stimulation pulses, such as voltage/current amplitude, pulse width, pulse train length, pulse spacing, coupling interval and/or other parameters. Although, in some examples, such parameters may be separate from the ATP therapy protocols. For example, LCP 100 may be preprogrammed with a number of ATP therapy protocol templates that define the ATP therapy protocol, but can be tailored via one or more parameter values, such as voltage/current amplitude, pulse width, pulse train length, pulse spacing, and/or other parameters. In such cases, the parameter values may have default values, which in some cases, may be replaced with different parameter values that are communicated to the LCP 100 from the MD 200.

Moreover, it is contemplated that the LCP 100 may determine whether the tachyarrhythmia is likely to be susceptible to ATP therapy based on a number of parameters. In some cases, these parameters may depend on the particular ATP therapy that is to be applied. It is contemplated that these parameter values may also have default values, which in some cases, may be replaced with different parameter values that are communicated to the LCP 100 from the MD 200. In some cases, these parameters may be part of an ATP therapy protocol, or may be separate from the ATP therapy protocol.

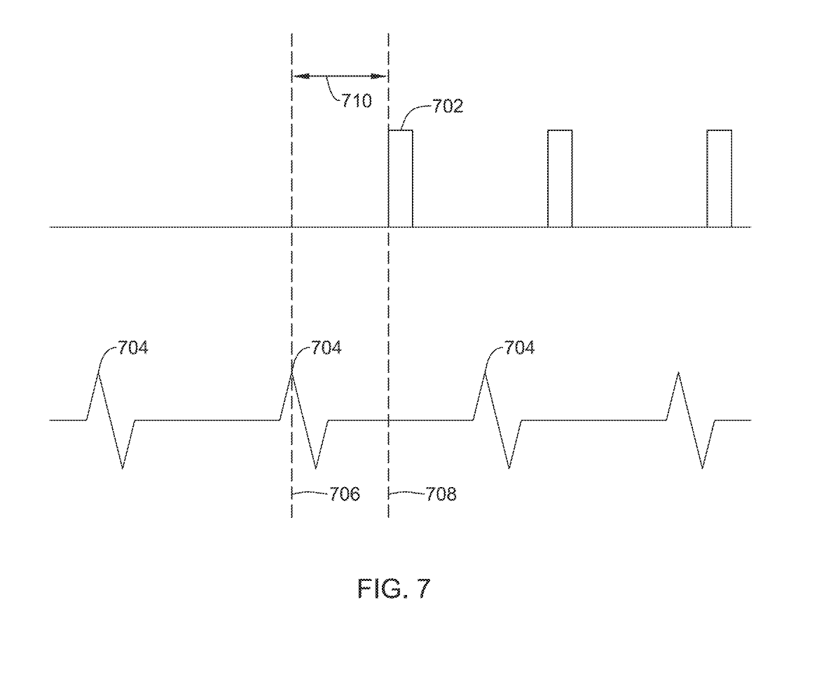

In some cases, when delivering electrical stimulation pulses of an ATP therapy, LCP 100 may time the electrical stimulation pulses based on the timing of the detected QRS complexes. For instance, as depicted in FIG. 7, QRS waves 704 may be used to identify the cardiac cycle of the heart. Generally, a heart is not able to contract in response to electrical stimulation just after a contraction of the heart (i.e. during a refectory period). After a certain time passes following a contraction, the cells of the heart may again be contracted in response to electrical stimulation. Accordingly, in order to deliver electrical stimulation therapy with a higher chance of causing a contraction of the heart or a high chance of terminating an arrhythmia, LCP 100 may wait to deliver electrical stimulation pulses until after the refractory period expires. In the example shown in FIG. 7, after LCP 100 senses a QRS wave 704 at time 706, LCP 100 may wait a predefined time period 710, known in the art as a coupling interval, before delivering an electrical stimulation pulse 702 at time 708. Time period 710 may be predefined such that electrical stimulation pulse 702 occurs during a non-refractory period of the heart (or at some other time that has a greater likelihood of terminating an arrhythmia). As such, LCP 100 may synchronize the ATP therapy relative to a sensed QRS complex. In some examples, LCP 100 may continue to deliver electrical stimulation pulses 702 at intervals 710 after detection of QRS complexes. However, in other examples, the interval between subsequent delivered electrical stimulation pulses 702 may be different than interval 710. In some examples, after stimulation pulse 702 at time 708 is delivered, subsequent stimulation pulses are delivered without regard to sensed QRS complexes.

In some cases, the LCP 100 may autonomously detect cardiac arrhythmias and apply ATP therapy if appropriate, independently of the MD 200. In other cases, there may be some cooperation between the LCP 100 and MD 200 to detect cardiac arrhythmias and deliver appropriate ATP therapy to the heart. For example, LCP 100 may receive information from MD 200 and/or LCP 100 may send information to MD 200. In some cases, MD 200 may make one or more determinations in order to detect cardiac arrhythmias and/or to apply ATP therapy if appropriate. As on example, MD 200 may determine occurrences of cardiac arrhythmias and, after determining an occurrence of a cardiac arrhythmia, may notify LCP 100 of the arrhythmia. LCP 100 may then determine whether the arrhythmia may be susceptible to ATP therapy and, if so, may deliver ATP therapy. Either device may then determine whether any delivered ATP therapy was successful in terminating the arrhythmia. In the case of LCP 100 determining that the ATP therapy was not successful in terminating the arrhythmia, LCP 100 may communicate a trigger message to MD 200 to deliver defibrillation therapy. In the case where MD 200 determines that ATP therapy was not successful in terminating the arrhythmia, MD 200 may determine to deliver defibrillation therapy.

In examples where LCP 100 and MD 200 cooperate, MD 200 may sense cardiac electrical signals and may communicate the received cardiac electrical signals to LCP 100. In other examples, MD 200 may send an indication of times when MD 200 sensed a peak or QRS complex in the cardiac electrical signals. From such received communications, LCP 100 may generate a beat rate. In other examples, MD 200 may determine a beat rate based on the cardiac electrical signals and only communicate the determined beat rate to LCP 100. LCP 100 may incorporate this received beat rate along with a locally generated beat rate in determining occurrences of arrhythmias. For example, LCP 100 may compare a received beat rate to a locally generated beat rate to confirm the beat rate, and compare the confirmed beat rate to one more beat rate thresholds. In some cases, the LCP 100 may use the slowest beat rate of the received beat rate and a locally generated beat rate, for comparison to the one or more beat rate thresholds. In some cases, these received cardiac electrical signals, received beat rate, and/or other received information may represent information that is not readily available to the LCP 100, such as atrial cardiac electrical signals, atrial beat rate, etc. In such examples, LCP 100 may be able to determine whether the ventricular beat rate is greater than the atrial beat rate to determine whether a tachyarrhythmia is likely to be susceptible to ATP therapy. MD 200 may send other signals or information as well, such as blood flow, blood pressure, acoustic information (e.g. heart sounds), and/or accelerometer information. MD 200 may communicate these additional signals to LCP 100, and LCP 100 may be configured to incorporate such information in determining occurrences of arrhythmias and/or whether an arrhythmia is likely to be susceptible to ATP therapy.

In some instances, LCP 100 may send information to MD 200. For example, LCP 100 may communicate sensed cardiac electrical signals to MD 200. MD 200 may process such received signals and send the processed signals back to LCP 100. In some cases, MD 200 may determine a beat rate based on the cardiac electrical signals received from LCP 100 and communicate the beat rate to LCP 100. LCP 100 may still determine occurrences of arrhythmias. However, some cases, MD 200 may make a determination as to the occurrences and/or types of arrhythmias.

In some cases, MD 200 may perform a susceptibility analysis on the cardiac electrical signals received from LCP 100 and communicate results to LCP 100. For example, MD 200 may determine a regularity parameter and determine whether the regularity parameter indicates that an arrhythmia is likely to be susceptible to ATP therapy. MD 200 may communicate this determination to LCP 100. In a similar manner, MD 200 may determine a morphology parameter and, if applicable, determine whether a ventricular beat rate is greater than the atrial beat rate. In such examples, LCP 100 may determine whether a tachyarrhythmia is likely to be susceptible to ATP therapy based at least in part on the received determinations of the MD 200. However, in some cases, MD 200 may make a determination as whether an arrhythmia is likely to be susceptible to ATP therapy, and may deliver that determination to the LCP 100.

In some cases, MD 200 may determine on its own whether ATP therapy delivered by LCP 100 was successful in terminating the tachyarrhythmia. In one example, LCP 100 may not communicate a message to MD 200 to deliver a defibrillation shock or an indication that the ATP therapy has failed. Rather, MD 200 may monitor the ATP therapy and determine on its own whether the ATP therapy has succeeded or not in terminating the tachyarrhythmia. Although LCP 100 may not communicate a trigger message to MD 200, LCP 100 may communicate a message that LCP 100 is about to, or has, performed ATP therapy. If MD 200 determines, for example after a predetermined period of time, that the ATP therapy has not terminated the tachyarrhythmia, MD 200 may determine to deliver a defibrillation shock. In other examples, MD 200 may receive a confirmation from LCP 100 that LCP 100 has also determined that the ATP therapy has failed to terminate the arrhythmia or that LCP 100 is finished attempting ATP therapy. In some examples, MD 200 may communicate an indication that it will deliver a defibrillation shock to LCP 100.

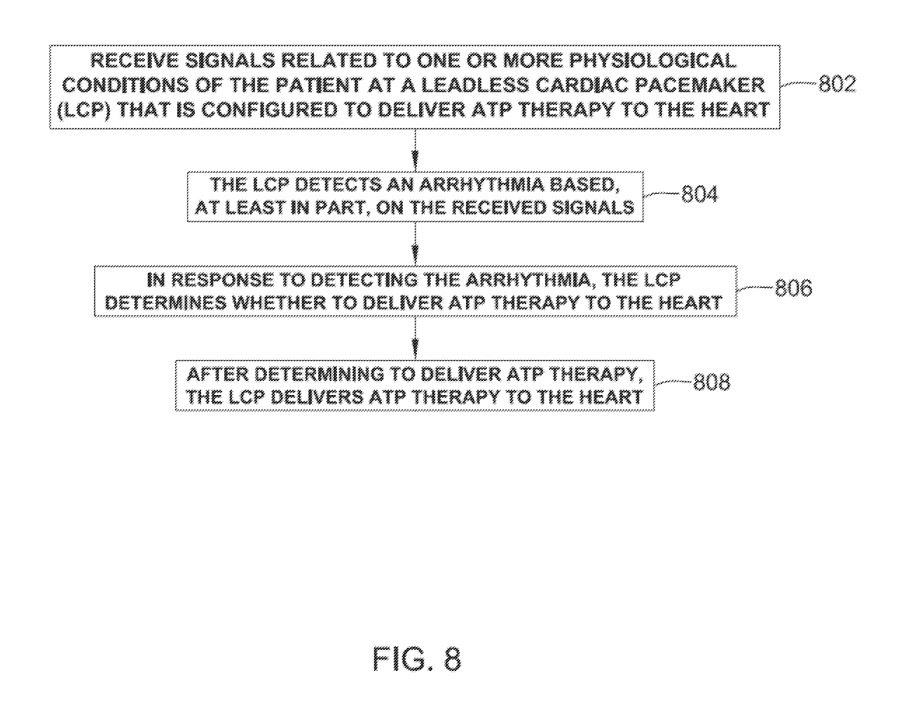

FIG. 8 is a flow diagram of an illustrative method that may be implemented by an implantable medical device, such as shown in FIGS. 1 and 2, or a medical device system such as shown in FIGS. 3 and 4. Although the method of FIG. 8 will be described with respect to LCP 100, the illustrative method of FIG. 8 may be performed using any suitable medical device or medical device system.

In some instances, a first implantable medical device, for instance LCP 100, may be implanted in a first chamber of a heart, such as an atrium or ventricle, and may receive signals related to one or more physiological conditions of a patient. The received signals may be signals received at the one or more electrodes of the LCP 100. The received signals may be physiological signals directly sensed by the one or more electrodes of the LCP 100, and/or signals communicated to the LCP from another medical device, such as MD 200.

The LCP 100 may also be configured to deliver ATP therapy to the heart, as shown at 802. LCP 100 may detect an arrhythmia based, at least in part, on the received signals, as shown at 804. In response to detecting an arrhythmia, LCP 100 may determine whether to deliver ATP therapy to the heart, as shown at 806. Finally, after determining to deliver ATP therapy, LCP 100 may deliver ATP therapy to the heart, as shown at 808.

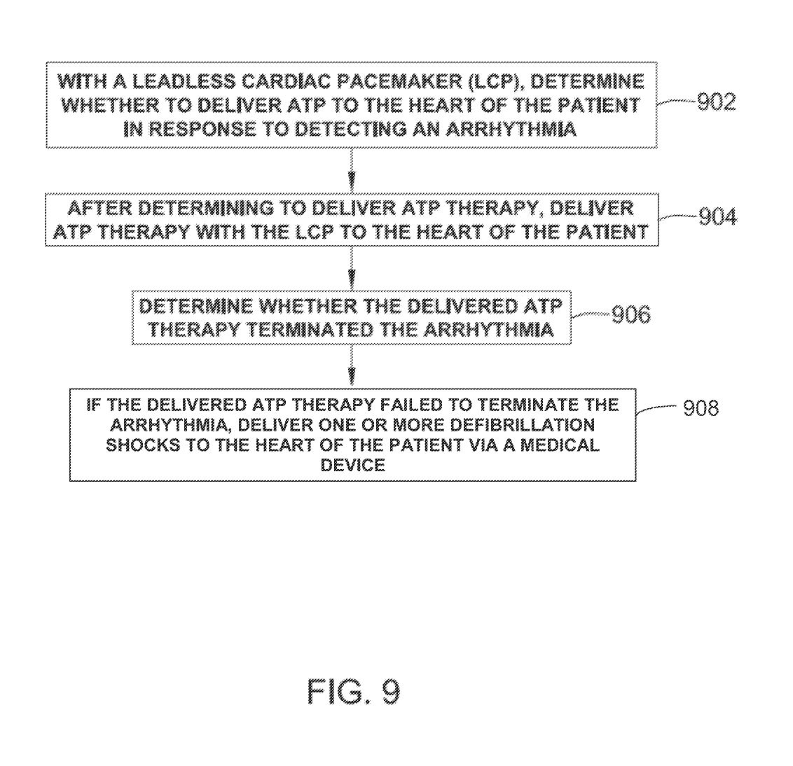

FIG. 9 is a flow diagram of an illustrative method that may be implemented by an implantable medical device, such as shown in FIGS. 1 and 2, or a medical device system such as shown in FIGS. 3 and 4. Although the method of FIG. 9 will be described with respect to LCP 100, the illustrative method of FIG. 9 may be performed by any suitable medical device or medical device system.

In some examples, a first implantable medical device, for instance LCP 100, may determine whether to deliver ATP to the heart of a patient in response to detecting an arrhythmia, as shown at 902. After determining to deliver ATP therapy, LCP 100 may deliver ATP therapy to the heart of the patient, as shown at 904. LCP 100 may determine whether the delivered ATP therapy terminated the arrhythmia, as shown at 906. If the delivered ATP therapy failed to terminate the arrhythmia, another medical device, for example MD 200, may deliver one or more defibrillation shocks to the heart of the patient, as shown at 908.

Those skilled in the art will recognize that the present disclosure may be manifested in a variety of forms other than the specific examples described and contemplated herein. For instance, as described herein, various examples include one or more modules described as performing various functions. However, other examples may include additional modules that split the described functions up over more modules than that described herein. Additionally, other examples may consolidate the described functions into fewer modules. Accordingly, departure in form and detail may be made without departing from the scope and spirit of the present disclosure as described in the appended claims.

What is claimed is:

1. A leadless cardiac pacemaker (LCP) comprising:
a housing;
one or more exposed electrodes;
a power source; and
a processing module, wherein the processing module is operatively coupled to the one or more exposed electrodes and the power source, the processing module is configured to:
receive signals related to one or more physiological conditions of a patient via one or more of the exposed electrodes;
detect an arrhythmia of a heart of the patient based, at least in part, on the received signals;
determine whether to deliver ATP therapy via one or more of the exposed electrodes in response to detecting an arrhythmia; and
deliver ATP therapy to the heart via one or more of the exposed electrodes if it is determined to deliver ATP therapy.

2. The LCP of claim 1, wherein the processing module is further configured to determine whether the delivered ATP therapy terminated the arrhythmia.

3. The LCP of claim 2, wherein the processing module is further configured to, after determining that the delivered ATP therapy failed to terminate the arrhythmia, communicate a trigger message to a medical device to deliver one or more defibrillation shocks, wherein the medical device is one of:
a leadless cardiac pacemaker;
an implantable cardioverter-defibrillator;
a subcutaneous implantable cardioverter-defibrillator; and
an external cardioverter-defibrillator.

4. A method for delivering anti-tachycardia pacing (ATP) therapy to a heart of a patient, the method comprising:
receiving signals related to one or more physiological conditions of the patient at a leadless cardiac pacemaker (LCP) that is configured to deliver ATP therapy to the heart;
the LCP detecting an arrhythmia based, at least in part, on the received signals;
in response to detecting the arrhythmia, the LCP determining whether to deliver ATP therapy to the heart; and
after determining to deliver ATP therapy, the LCP delivering ATP therapy to the heart.

5. The method of claim 4, further comprising after delivering ATP therapy, the LCP determining whether the delivered ATP therapy terminated the arrhythmia.

6. The method of claim 5, further comprising:
if the LCP determines that the delivered ATP therapy failed to terminate the arrhythmia, the LCP communicating a trigger message from the LCP to a medical device, wherein in response to receiving the trigger message, the medical device delivering one or more defibrillation shocks to the heart.

7. The method of claim 4, further comprising:
after another medical device determines that the delivered ATP therapy failed to terminate the arrhythmia, delivering one or more defibrillation shocks to the heart with the another medical device.

8. The method of claim 4, wherein the signals received by the LCP include an ECG signal sensed by the LCP.

9. The method of claim 8, further comprising processing the received ECG signal to identify one or more of an atrial rate parameter, a ventricle rate parameter, a morphology parameter, a rhythm parameter.

10. The method of claim 9, wherein the LCP, in determining whether to deliver ATP therapy to the heart, determines if the ventricle rate parameter is above a predetermined threshold.

11. The method of claim 9, wherein the LCP, in determining whether to deliver ATP therapy to the heart, determines if the ventricle rate parameter is above a predetermined threshold indicating a high heart rate, and the rhythm parameter is above a predetermined threshold indicating a sufficiently regular heart rhythm.

12. The method of claim 9, wherein the LCP, in determining whether to deliver ATP therapy to the heart, determines if the morphology parameter indicates a monomorphic arrhythmia and if the ventricle rate parameter is greater than the atrial rate parameter.

13. The method of claim 4, wherein at least some of the received signals are physiological signals sensed by the LCP, and at least some of the received signals are signals communicated to the LCP from another medical device.

14. An implantable medical device system for delivering anti-tachycardia pacing (ATP) therapy to a heart of a patient, the system comprising:
a leadless cardiac pacemaker (LCP) implanted in or proximate the heart of the patient; and
a medical device implanted within the patient and communicatively coupled to the LCP;
wherein the LCP is configured to:
sense cardiac electrical signals;
determine whether to deliver ATP therapy based at least in part on the sensed cardiac electrical signals;
after determining to deliver ATP therapy based at least in part on the sensed cardiac electrical signals, deliver ATP therapy to the heart of the patient; and
wherein the medical device is configured to deliver one or more defibrillation shocks to the heart of the patient.

15. The system of claim 14, wherein the LCP, when determining whether to deliver ATP therapy, determines whether an arrhythmia having predetermined characteristics is present, and if present, determines to deliver ATP therapy.

16. The system of claim 14, wherein the LCP is further configured to determine whether delivered ATP therapy terminated the arrhythmia, and if not, to communicate a trigger signal to the medical device; and
wherein the medical device is configured to deliver one or more defibrillation shocks to the heart of the patient in response to receiving the trigger signal from the LCP.

17. The system of claim 14, wherein the medical device is further configured to:
determine whether delivered ATP therapy terminated the arrhythmia; and
deliver one or more defibrillation shocks to the heart of the patient in response to determining that the delivered ATP therapy failed to terminate the arrhythmia.

18. The system of claim 14, wherein:
the medical device is further configured to sense cardiac electrical signals and communicate the sensed cardiac electrical signals to the LCP; and
the LCP is configured to determine to deliver ATP therapy based at least in part on cardiac electrical signals sensed by the LCP and the cardiac electrical signals communicated by the medical device.

19. The system of claim 14, wherein the LCP is further configured to communicate sensed cardiac electrical signals to the medical device, and wherein the medical device is configured to process the received cardiac electrical signals and communicate one or more processed cardiac signals to the LCP.

20. The system of claim 14, further comprising an external support device, wherein the LCP and implantable medical device are configured for intra-body communication, and wherein the implantable medical device and external support device are configured for extracorporeal communication, and wherein the implantable medical device relays communications from the LCP to the external support device and communications from the external support device to the LCP.
